(19) United States
(12) Patent Application Publication
(10) Pub. No.: US 2017/0304325 A1
(43) Pub. Date: Oct. 26, 2017

(54) TREATMENT OF RHEUMATOID ARTHRITIS

(71) Applicant: Seven to Stand, Inc.

(72) Inventors: Fabrizio De Silvestri, Terni (IT); Edoardo Romani, Terni (IT)

(73) Assignee: Seven to Stand, Inc.

(21) Appl. No.: 15/376,953

(22) Filed: Dec. 13, 2016

Related U.S. Application Data
(63) Continuation of application No. 14/794,856, filed on Jul. 9, 2015, now abandoned.

(30) Foreign Application Priority Data
Aug. 20, 2014 (IT) PCT/IT14/000223

(51) Int. Cl. A61K 31/65; A61K 31/522; A61K 31/40; A61K 31/593; A61K 31/00

(52) U.S. Cl. CPC A61K 31/65; A61K 31/593; A61K 31/00; A61K 31/40; A61K 31/522

(57) ABSTRACT

A composition for treating chronic inflammation, including an antibiotic, a lipophilic potentiating agent, a guanosine analog antiviral agent, and Vitamin D in synergistically effective amounts. A method of treating chronic inflammation, by administering a synergistically effective amount of a composition including an antibiotic, a lipophilic potentiating agent, a guanosine analog antiviral agent, and Vitamin D to an individual suffering from chronic inflammation, and treating chronic inflammation. A method of reducing and/or eliminating symptoms of chronic inflammation, by administering a synergistically effective amount of a composition including an antibiotic, a lipophilic potentiating agent, a guanosine analog antiviral agent, and Vitamin D to an individual suffering from chronic inflammation, and reducing and/or eliminating the individual's symptoms of chronic inflammation.

### BACKGROUND OF THE INVENTION

### 1. Technical Field

The present invention relates to compositions and methods of treating arthritis and chronic inflammation. More specifically, the present invention relates to compositions and methods of treating rheumatoid arthritis.

### 2. Background Art

Rheumatoid arthritis (RA) is a chronic inflammatory polyarthritis, spondylitis, and progressive autoimmune disease of unknown etiology and at the expense of the synovial joints (i.e. the small joints in the hands and feet). RA is different than osteoarthritis in that it affects the synovial membrane and cartilage of joints and occurs less frequently and at a younger age than osteoarthritis.

RA is a chronic disease that is estimated to affect between 0.3 and 1.0% of the world population. The disease is much more common in women, who are generally four times more affected than men. Pain, fatigue, and depression accompany the disease, characterized by progressive damage to articular anatomy resulting in disability, to date clinically assessed with the EDSS (Expanded Disability Status Scale), decreased quality of life, and decreased life expectancy. RA is a chronic disease, although spontaneous remission can occur in rare cases after conducting a pregnancy: during pregnancy the hormonal changes can temporarily block the inflammatory state and in rare cases there is a definite remission.

RA affects the joints, making it difficult for the sliding of the bone joints, leading to degenerative medical condition that manifests itself at the level of the cartilage and the synovial membrane that is in contact with the area exposed to inflammation.

The exact cause remains unknown, but there is thought to be a genetic influence from the association with MHC-II antigens and in particular HLA-DR1 and HLA-DR4. Approximately 80% of RA patients are positive for the rheumatoid factor (RF), an autoantibody that can be detected in the blood that is generally not detectable in the blood of unaffected individuals. RF can be of the type IgM, IgA, IgG, IgE, or IgD. Elevated levels can indicate disease severity. Another diagnostic marker is anti-cyclic citrullinated peptides, which seem to have a role in the early diagnosis of the disease. It is also possible that the Epstein-Barr virus or Mycobacterium tuberculosis have a role in causing RA. RF has been found in Epstein-Barr virus and Parvovirus infected individuals. It is therefore possible that a microorganism causes the inflammatory reaction in the immune system, which is only for a limited period of time, and usually the result of diseases such as mononucleosis (often confused with disease states such as seasonal influenza virus). Superantigens may be involved as well as self-antigens (collagen, proteoglycans, RF, and citrullinated proteins) in playing a role in the chronicity of the disease.

In the pathogenesis of RA, the synovium is a membrane of mesenchymal source formed by synoviocytes type 1 (macrophage) and type 2 (fibrinoid) and in disease undergoes hyperplasia and hypertrophy. It grows in thickness (it usually consists of two or at most three layers of cells, whereas in the disease it becomes seven layers or more) and is formed so that the pannus begins peripherally to erode the bone not covered by cartilage (bare bone).

The typical symptoms are pain, swelling, and functional impotence of the joints, typically the proximal interphalangeal (IFP) and/or metacarpophalangeal (MCP). These symptoms are related to the biological circadian rhythm. In fact, in healthy subjects, the level of pro-inflammatory cytokines (such as TNF-α and interleukin IL-6) increases during the night to reach the peak in the early morning. In addition, cytokines activate the hypothalamic-pituitary-adrenal axis, inducing metabolic changes typical of chronic inflammatory conditions of RA. Therefore, phenomena such as stiffness, pain, and swelling vary throughout the day, occurring mostly during the early hours of the morning.

The inflammation can also affect the tendons and there are different clinical manifestations, “finger swan neck” with hyperextension of the proximal interphalangeal joint (IFP) and flexion of the distal interphalangeal joint (IFD); “finger on the button in the loop” or en boutonniere with flexion and hyperextension of the IFP IFD; or “hammer toe” with an extensor tendon injury that can result in fixed flexion of the distal phalanx. Another feature is the Baker's cyst at the level of the popliteal fossa which can break, creating bruising.

The disease is systemic so it can involve other organs and systems. Typical are rheumatoid nodules, superficial or deep, which can also occur in the lung. There may therefore be pulmonary fibrosis, pleurisy, and pleuropericarditis. In the cardiac system, there can be an acceleration of atherosclerosis of the coronary arteries. At eye level there can be xerophthalmia, uveitis, and scleritis. The iatrogenic pathologies are different while complications of RA are amyloidosis and osteoporosis. In addition, there is an increase in ESR (erythrocyte sedimentation rate), CRP (C-reactive protein), fever, and general malaise.

There are four clinical variants of RA. Caplan's syndrome is characterized by a lung involvement, with pulmonary nodules, linked to exposure to asbestos, silica, and charcoal. The rheumatoid nodules in the presence of the above agents increase in size and may coalesce and form of the excavations. Felty's syndrome adds to the typical manifestations of RA the presence of splenomegaly and leukopenia. Still's disease has an association with the presence of a macular rash high fever, but with a generally fleeting and non-erosive polyarthritis. Finally, Malignant Rheumatoid Arthritis is a particularly severe form with vasculitic involvement widespread and important bony erosions.

The state of the disease can be identified by analyzing the type of injury to the patient:

Stage 1: There is an infiltration of CD4+ lymphocytes and macrophages. Macroscopically symmetric swelling can be seen with no redness. There are systemic symptoms and rheumatoid nodules. An increase is noted in the circulation of inflammatory markers and rheumatoid factor.

Stage 2: Inflammation and synovial and endothelial proliferation (angiogenesis and pannus formation) are noticed. The anomaly is seen on ultrasound as hypoechoic areas; on the contrary hyperplastic areas are hyperechoic. There are also erosions of the bone, resorption of cartilages, and tendon ruptures. The bone changes are seen in X-rays and even better in ultrasound. From this stage, the synovial hyperplasia is irreversible.

Stage 3: There are bone deformities, dislocations, and fibrosis is evident. The course is varied and generally characterized by periods of exacerbation and remission. There are milder forms that respond well to current treatments and serious forms which run without remission, leading to ankylosis and serious functional impairment. In many cases, the disease is not serious because it would endanger the life, but because, by preventing the proper use of the limbs, especially the hands and feet, involves a high degree of debilitation, currently evaluated clinically with the EDSS (Expanded Disability Status Scale page), where those affected find it difficult not only in every day movements but also in the care of one's self.

In general, an early diagnosis is important because in the very first months of the onset of disease substantial damage is observed and is irreversible. Also, in the first two years of disease after diagnosis, the damage is particularly severe. The impairment of the joints leads to a limitation of mobility that can result in disability and subsequent premature death.

Clinical criteria requires the presence of at least four of the following symptoms to formulate a probable diagnosis of RA: morning stiffness lasting at least one hour, arthritis at the level of three or more joints, arthritis of the joints of the hand, symmetrical arthritis, cutaneous rheumatoid nodules, a positive test for rheumatoid factor, or radiological changes. The most useful tests for the diagnosis of RA include anticitrulline antibodies (CCP), rheumatoid factor, ESR, and CRP. Other manifestations are not pathognomonic for RA and include carpal tunnel syndrome (because the involvement of MCF (macrophage chemotactic factor) damages the median nerve), vasculitis, Sjogren's syndrome, amyloidosis, and alterations in the pleura and pericardium.

Presently, there is no known pharmacological treatment that involves a final shutdown of inflammation nor a restoration of joint function. At a mechanical level, physical therapy can be used to alleviate the effects in advanced stages by a surgery to remove the too exuberant pannus (synoviectomy), practice arthroplasty, and arthrodesis. In addition, the eventual recovery of joint function can be assessed only in the surgical phase of the submissive disease. The interventions are invasive and take place in the reconstruction of bone and cartilage with the introduction of prosthesis. In general, this does not solve the problem to the joints and can become a potential effect of risk in the event of reactivation of the disease. Furthermore, they should be subject to periodic inspections and the success of the intervention is not guaranteed in general.

The drugs used in the treatment of RA are varied, the properties and their side effects in the short and long term are now known as the 90% of them have been used for many years. The therapeutic approach has changed considerably over time as the current drugs tend to attack the disease upon its detection, whereas early drugs were used as a last resort.

The drugs can be divided in to two types, “symptomatic” and “background”. Among the “symptomatic” drugs are: aspirin, acetaminophen and other non-steroidal anti-inflammatory drugs (NSAIDs), and corticosteroids (anti-inflammatory drugs). These are drugs that contain the pain and inflammation but do not change the evolution of the disease. The “background” drugs include those substances that modify the clinical course of the disease and slow down in time the evolution of the anatomical damage of the joints: gold salts, antimalarials, d-penicillamine, sulfalazina, immunosuppressants (methotrexate, cyclosporin A, ARAVA® (Sanofi, leflunomide), as well as biologics or anti-tumor necrosis factor (TNF-α). Some of these treatments are currently used on people with RA after traditional treatments have virtually failed.

Currently, for mild forms of initial RA, anti-inflammatory and antirheumatic drugs are prescribed. For the most aggressive and advanced stages of RA, methotrexate is prescribed. Patients treated with methotrexate should be monitored constantly because of the high toxicity of the drug at the level of various organs including bone marrow, liver, kidneys, and lungs. In case of occurrence of adverse effects, it is necessary to reduce the dosage and co-administer with folinic acid or discontinue therapy.

Leflunomide is used in moderate-to-severe RA and inhibits pyrimidine synthesis. It is the alternative to methotrexate in resistant patients. Side effects can include liver damage, lung disease, and immunosuppression.

Biologics (monoclonal antibodies) act more selectively and specifically in RA and can include adalimumab (TNF-α inhibitor), rituximab (B cell destroyer), infliximab (TNF-α inhibitor), and etanercept (TNF inhibitor). These are found to have the same side effects as methotrexate. In some cases, patients have developed Hodgkin's disease, although the cause-effect correlation is yet to be ascertained. Other side effects, especially with etanercept and other immunosuppressants (methotrexate and corticosteroids), include the occurrence of tumors and malignancies.

Recent studies have also found that some people with RA have had relief from cannabis use and the drug form nabiximols (SATIVEX®, GW Pharma) that is a cannabis extract containing tetrahydrocannabinol and cannabidiol.

There remains a need for a treatment for RA that is able to target the cause of the disease and eliminate side effects as well as provide improvement of disability.

### SUMMARY OF THE INVENTION

The present invention provides for a composition for treating chronic inflammation, including an antibiotic, a lipophilic potentiating agent, a guanosine analog antiviral agent, and Vitamin D in synergistically effective amounts.

The present invention also provides for a method of treating chronic inflammation, by administering a synergistically effective amount of a composition including an antibiotic, a lipophilic potentiating agent, a guanosine analog antiviral agent, and Vitamin D to an individual suffering from chronic inflammation, and treating chronic inflammation.

The present invention provides for a method of reducing and/or eliminating symptoms of chronic inflammation, by administering a synergistically effective amount of a composition including an antibiotic, a lipophilic potentiating agent, a guanosine analog antiviral agent, and Vitamin D to an individual suffering from chronic inflammation, and reducing and/or eliminating the individual's symptoms of chronic inflammation.

### DETAILED DESCRIPTION OF THE INVENTION

The present invention generally provides for compositions and methods of treating chronic inflammation, and especially rheumatoid arthritis (RA).

“Chronic inflammation” as used herein, refers to any type of inflammation characterized by a slow onset with a long duration, an infiltration of monocytes, macrophages, and/or lymphocytes, severe and progressive tissue injuries or fibrosis, or subtle signs of inflammation. Preferably, the chronic inflammation is due to RA. The chronic inflammation can be any that is linked to a lipid metabolic syndrome (i.e. any disorder of energy utilization and storage and occurring with obesity, elevated blood pressure, elevated fasting blood glucose, high serum triglycerides, and low high-density lipoprotein (HDL) levels).

“Rheumatoid arthritis” as used herein can be any type of chronic inflammatory polyarthritis, and any clinical variant described above such as Caplan's syndrome, Felty's syndrome, Still's disease, or Malignant Rheumatoid Arthritis. The RA can be at any stage.

“Recovering mobility” as used herein refers to recovering at least some, and preferably most or all, use of limbs and extremities, especially hands and feet, such that an individual can move without as much restriction and/or pain as previously as well as with more control.

The composition of the present invention includes an antibiotic, a lipophilic potentiating agent, a guanosine analog antiviral agent, and Vitamin D in synergistically effective amounts. More preferably, the composition includes a tetracycline antibiotic, a lipophilic statin, a guanosine analog antiviral agent, and Vitamin D in synergistically effective amounts. Even more preferably, the composition includes minocycline, atorvastatin, acycloguanosine, and Vitamin D3. Preferably, the composition is a pharmaceutical composition including pharmaceutically acceptable excipients.

The antibiotic can be any suitable antibiotic and is preferably a tetracycline antibiotic. Most preferably, the tetracycline antibiotic is minocycline ((2E,4S,4aR,5aS,12aR)-2-(amino-hydroxy-methylidene)-4,7-bis(dimethylamino)-10,11,12a-trihydroxy-4a,5,5a,6-tetrahydro-4H-tetracene-1,3,12-trione). Minocycline is long-acting and has a longer half-life than other tetracyclines. Any other equivalent forms can be used. Other tetracycline antibiotics with the same function that can be used include, but are not limited to, tetracycline, chlortetracycline, oxytetracycline, demeclocycline, methacycline, tigecycline, or doxycycline. In general, tetracycline antibiotics act to inhibit protein synthesis and the binding of aminoacyl-tRNA to the mRNA-ribosome complex. The dose of the tetracycline antibiotic can be from 25 mg to 500 mg, every 12 hours, depending on age and weight. Preferably, the dose is from 50 mg to 100 mg. Most preferably, the dose is 100 mg. It should be understood that a lower dose can be used because of the synergy of the components.

The antibiotic, and especially minocycline, can act as a catalyst at the cellular level, crossing the blood-brain barrier, a role that is more enhanced by the use of acycloguanosine, suitable to permanently remove the residues present at the intracellular level of an alleged mononucleosis (diagnosed or not) and related strains. These strains have probably remained inert in the body and matured as a result of hormonal changes or stressors. In women, this manifests itself during menopause or during the period of greatest fertility, which would also explain the rare cases of complete remission after pregnancy. In men, consequently, this could manifest demurring a period of testosterone deficiency with a prevalence of 17beta estradiol.

The lipophilic potentiating agent can be any suitable agent that is able to cross the blood-brain barrier. Preferably, the lipophilic potentiating agent is a lipophilic statin. The lipophilic statin is preferably atorvastatin (LIPITOR®, Pfizer) ((3R,5R)-7-[2-(4-Fluorophenyl)-3-phenyl-4-(phenylcarbamoyl)-5-propan-2-ylpyrrol-1-yl]-3,5-dihydroxyheptanoic acid). Atorvastatin is a competitive inhibitor of HMG-CoA reductase, reducing cholesterol. Lipophilic statins are effective at crossing the blood-brain barrier into the CNS. Any other equivalent forms can be used. Other lipophilic statins with the same function that can be used include, but are not limited to, lovastatin, simvastatin, cerivastatin, fluvastatin, and mevastatin. The role of the lipophilic statin is to facilitate the restoration of the viscosity of synovial fluid in RA that is characterized by thickening caused by dysfunctional metabolism secondary to lipid accumulation. This thickening generates joint inflammation. The dose of the lipophilic statin can be 5 mg to 40 mg every 12 hours, depending on age and weight. Most preferably, the dose is 20 mg, or even 10 mg in order to avoid rhabdomyolysis. It should be understood that a lower dose can be used because of the synergy of the components.

The guanosine analog antiviral agent can be any suitable guanosine analog antiviral agent such as, but not limited to, acycloguanosine, valacyclovir, penciclovir, famciclovir, and ganciclovir. Preferably, the guanosine analog antiviral agent is acycloguanosine (2-Amino-1,9-dihydro-9-((2-hydroxyethoxy)methyl)-6H-purin-6-one), also known as aciclovir. Aciclovir is converted to its monophosphate by viral thymidine kinase, and subsequently to its triphosphate (ACV-TP) by host cell kinases. ACV-TP competitively inhibits and inactivates viral DNA polymerases and prevents further DNA synthesis. It is most commonly used against herpes simplex virus types I and II, varicella zoster virus, Epstein-Barr virus, and cytomegalovirus. In general, the role of the guanosine analog antiviral agent is to eliminate latent viral potential by eradicating viral caspids. The dose of the guanosine analog antiviral agent can be 50 mg to 400 mg, every 12 hours, depending on age and weight. Preferably, the dose of the guanosine analog antiviral agent is 200 mg. It should be understood that a lower dose can be used because of the synergy of the components.

The Vitamin D (i.e. D group Vitamin) is a pro-hormone, and can be Vitamin D2 (ergocalciferol), which is of plant origin or Vitamin D3 (cholecalciferol), which is of animal origin. Most preferably, the Vitamin D is Vitamin D3. Vitamin D3 is a fat-soluble secosteroid that enhances intestinal absorption of calcium, iron, magnesium, phosphate, and zinc. Vitamin D3 is a pro-hormone, is able to fix calcium in the bones, thus facilitating the recovery of damaged tissue by arthritis. The thickening of the synovial pannus means that this acts as a sponge, preventing the passage of calcium, which is fixed in locations surrounding the joint, making the same dysfunctional. Therefore, the use of a D Vitamin in combination with the other components of the composition ensures that the calcium is absorbed by the right target and not dispersed or fixed at the level of cartilage. The dose of the Vitamin D can be 50 I.U. to 1000 I.U., every 12 hours, depending on age and weight, and preferably 400 I.U. to 1000 I.U. Most preferably, the dose is 400 I.U. It should be understood that a lower dose can be used because of the synergy of the components.

Preferably, the composition is in a single oral dosage form, such as a pill, capsule, or tablet, with each of the antibiotic, antifungal agent, and lipophilic potentiating agent contained therein or within a coating. Different combinations or each component can be included within the oral dosage form or within its coating. The composition can be tailored to provide different release profiles as needed or desired for a particular patient, such as, but not limited to, sustained release, prolonged release, or immediate release. The antibiotic, lipophilic potentiating agent, guanosine analog antiviral agent, and Vitamin D3 can each have the same release profiles or different release profiles. However, other dosage forms and routes can be used as detailed below. Preferably, the dosage form is gastroresistant.

The preferred combination of components in the composition is 100 mg minocycline, 20 mg atorvastatin, 200 mg acycloguanosine, and 400 IU Vitamin D3 in a single dosage form.

Preferably, the composition is administered once every 12 hours, considering the half-lives of the components. Other times of administration can be used depending on the dosage. While beneficial effects can be experienced upon taking the first dose, it is preferred that an individual continue treatment for days, weeks, months, and/or years. Treatment can be 45 days or longer. It is preferable that the treatment is a one-time treatment, as it eliminates the root causes of chronic inflammation. However, repeat treatments can be performed when necessary.

The four components of the composition (antibiotic, lipophilic potentiating agent, guanosine analog antiviral agent, and Vitamin D) produce a potentiated effect as opposed to their effect alone because they act synergistically together. This can result in a lower dose of each component required to be effective and reduced side effects. In the examples below, no noteworthy side effects were experienced. The combination of an antibiotic and the lipophilic potentiating agent implement the elimination and eradication of capsids immortalized by a consequent inflammatory process and induce a regenerative process of injured areas. The combination of minocycline, atorvastatin, acycloguanosine, and Vitamin D3 was chosen because of the low toxicity of each drug, their high ability to act at sinovial and joint levels, the fact that all have the same half-life, and their ability to interact synergistically with each other. If administered in conjunction with a suitable excipient, atorvastatin's ability to penetrate synovial tissues enhances the immunomodulating effects of minocycline, as well as the antiviral properties of acycloguanosine and Vitamin D3's ability to improve joint elasticity and ability to restore cartilage. This interaction promotes a better lipid metabolism that restores the natural synovial thickness. A synergistic effect is also produced between the components and the antiviral to reduce the presence of viruses, bacteria an/or fungi, or the proliferation of the same in an environment overloaded from antibiotics, or already infested by candida albicans, herpes viruses, etc. Often the presence of fungi is high due to previous infections brought upon by the imbalance caused by the massive use of corticosteroids, immunosuppressants and the like, which alone would be eligible to trigger a mechanism of excessive proliferation of the same that in turn, can trigger systemic intoxication and amplify the joint inflammatory process. Acycloguanosine was chosen for its half-life and because it acts primarily in the lipophilic environment.

The compounds of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of treating chronic inflammation, by administering a synergistically effective amount of a composition including an antibiotic, a lipophilic potentiating agent, a guanosine analog antiviral agent, and Vitamin D3 to an individual suffering from chronic inflammation, and treating chronic inflammation. Preferably, the chronic inflammation is due to RA. Preferably, the antibiotic is a tetracycline antibiotic and the lipophilic potentiating agent is a lipophilic statin. Most preferably, the tetracycline antibiotic is minocycline, the lipophilic statin is atorvastatin, and the guanosine analog antiviral agent is acycloguanosine. The antibiotic, lipophilic potentiating agent, and guanosine analog antiviral agent can also be any of those described above. Preferably, the composition is administered in a single dosage form orally once every 12 hours. Preferably, the single dosage form includes 100 mg minocycline, 20 mg atorvastatin, 200 mg acycloguanosine, and 400 IU Vitamin D3. Treatment can last 45 days or longer.

Treating the chronic inflammation, especially in RA, can include softening accumulations of synovial pannus, significantly reducing amounts of synovial pannus (synovial lining) in order to restore the natural synovial thickness, as well as restoring articulation and functional recovery of limbs affected by the chronic inflammation (especially the hands and feet). Pain, stiffness in the joints, and swelling can be reduced and/or eliminated. The antibiotic acts to remove residues at the intracellular level of mononucleosis and related strains. The lipophilic potentiating agent acts to restore the viscosity of synovial fluid and reduce thickened synovial membrane, thereby reducing joint inflammation. The guanosine analog antiviral agent acts to eliminate latent viral potential by eradicating viral caspids. The Vitamin D acts to fix calcium in the bones instead of being absorbed in the synovial pannus. Rheumatoid nodules can be absorbed. The method can also reduce an individual's EDSS value due to the functional recovery. The combination of the antibiotic, guanosine analog antiviral agent, and the lipophilic potentiating agent act to eliminate and eradicate capsids immortalized by inflammatory processes and induce a regenerative process of injured areas. This action contributes to a combined synergistic action with Vitamin D, which induces a functional recovery of the joint characterized by a restoration of the correct thickness of synovial tissues and increased nutrient supply to the cartilage itself.

The present invention also provides for a method of reducing and/or eliminating symptoms of chronic inflammation, by administering a synergistically effective amount of a composition including an antibiotic, a lipophilic potentiating agent, a guanosine analog antiviral agent, and Vitamin D to an individual suffering from chronic inflammation, and reducing and/or eliminating the individual's symptoms of chronic inflammation. Preferably, the chronic inflammation is due to RA. The composition can be any of those described above. Preferably, the composition is administered in a single dosage form orally once every 12 hours. Preferably, the single dosage form includes 100 mg minocycline, 20 mg atorvastatin, 200 mg acycloguanosine, and 400 IU Vitamin D3. Preferably, the symptoms reduced and/or eliminated include stiffness in the joints, pain, and swelling. Any combination or all of these symptoms can be improved with this method.

The present invention provides for a method of recovering mobility, by administering a synergistically effective amount of a composition including an antibiotic, a lipophilic potentiating agent, a guanosine analog antiviral agent, and Vitamin D to an individual suffering from chronic inflammation, and recovering mobility. Preferably, the chronic inflammation is due to RA. The composition can be any of those described above. Preferably, the composition is administered in a single dosage form orally once every 12 hours. Preferably, the single dosage form includes 100 mg minocycline, 20 mg atorvastatin, 200 mg acycloguanosine, and 400 IU Vitamin D3. Recovering mobility can include recovering the ability to move joints, especially in the hands and feet, and recovering motor control, and generally recovering function of the body. Recovering mobility can also include reducing and/or eliminating stiffness in the joints, pain, and swelling. Any or all of these recoveries can occur with this method. By performing this method, an individual can regain use of their hands and feet to do everyday activities such as walking and turning door knobs.

The composition of the present invention is advantageous in that because it effectively reduces inflammation, other anti-inflammatory drugs are not necessary and thus side effects of NSAIDS and other anti-inflammatory drugs can be avoided. In the examples below, none of the components of the composition of the present invention interacted adversely with any drug already used by patients and none of them had a relapse during treatment. It is noted, however, it is preferable to suspend current treatments with other drugs, especially methotrexate (chemotherapeutic or immunosuppressive agents and in general) and immunomodulating treatments.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

A female (M.D.) (40 years old) was suffering from RA for over 20 years. She previously took methotrexate and ENBREL® without any real effect in remission of the disease. After administration of the composition of the present invention (100 mg minocycline, 20 mg atorvastatin, 200 mg acycloguanosine, and 400 IU Vitamin D3 orally every 12 hours), she has reported a significant decrease in pain. A reduction in the thickness of the synovial lining and gradual absorption of rheumatoid nodules have been objectively noted. She showed a significant functional recovery as early as the first week of dosing with total absence of pain despite the lack of intake of other substances. To date, she has had a significant improvement in her quality of life, having recovered a great deal of autonomy.

### EXAMPLE 2

A female (U.V.) was suffering from RA for over 25 years. She previously took prednisone, arava, and methotrexate without any real effect in remission of the disease. After administration of the composition of the present invention (100 mg minocycline, 20 mg atorvastatin, 200 mg acycloguanosine, and 400 IU Vitamin D3 orally every 12 hours), she has reported a significant decrease in pain. A reduction in the thickness of the synovial lining, and gradual absorption of rheumatoid nodules have been objectively noted. She showed a significant functional recovery as early as the first week of dosing with total absence of pain despite the lack of intake of other substances. To date, she has had a significant improvement in quality of life, having recovered a significant amount of autonomy: She can open and close the hands without pain.

### EXAMPLE 3

A male (P.P.) was suffering from RA for over 3 years. He previously took prednisone and methotrexate without any real effect in remission of the disease. After administration of the composition of the present invention (100 mg minocycline, 20 mg atorvastatin, 200 mg acycloguanosine, and 400 IU Vitamin D3 orally every 12 hours), he has reported a significant decrease in pain, and absorption of rheumatoid nodules has been objectively noted. He showed a significant functional recovery as early as the first week of dosing with total absence of pain. To date, he has had a significant improvement in quality of life with a totally lack of articular pain.

### EXAMPLE 4

A female (S.M.) was suffering from RA for over 22 years. She previously took prednisone, methotrexate and ENBREL® without any real effect in remission of the disease. After administration of the composition of the present invention (100 mg minocycline, 20 mg atorvastatin, 200 mg acycloguanosine, and 400 IU Vitamin D3 orally every 12 hours), she reports no pain. A reduction in the synovial lining, and gradual absorption of rheumatoid nodules has been noted. She showed a significant functional recovery as early as the first week of dosing with total absence of pain despite the lack of intake of other substances. To date, she has had a significant improvement in quality of life, having recovered significant autonomy.

### EXAMPLE 5

A male (N.I.) was suffering from RA for over 5 years. He previously took deltacortene prednisone, methotrexate and ENBREL® without any real effect in remission of the disease. After administration of the composition of the present invention (100 mg minocycline, 20 mg atorvastatin, 200 mg acycloguanosine, and 400 IU Vitamin D3 orally every 12 hours), he experienced disappearance of pain and gradual absorption of rheumatoid nodules. He showed a significant functional recovery as early as the first week of dosing with total absence of pain despite the lack of intake of other substances. To date, he has had a significant improvement in quality of life, having no post-treatment complaints of articular pain.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition for treating chronic inflammation, comprising an antibiotic, a lipophilic potentiating agent, a guanosine analog antiviral agent, and Vitamin D in synergistically effective amounts.

2. The composition of claim 1, further defined as a tetracycline antibiotic, a lipophilic statin, a guanosine analog antiviral agent, and Vitamin D in synergistically effective amounts.

3. The composition of claim 2, wherein said tetracycline antibiotic is chosen from the group consisting of minocycline, tetracycline, chlortetracycline, oxytetracycline, demeclocycline, methacycline, tigecycline, and doxycycline.

4. The composition of claim 2, wherein said tetracycline antibiotic is present in the amount of 25 mg to 500 mg.

5. The composition of claim 2, wherein said lipophilic statin is chosen from the group consisting of atorvastatin, lovastatin, simvastatin, cerivastatin, fluvastatin, and mevastatin.

6. The composition of claim 2, wherein said lipophilic statin is present in the amount of 5 mg to 40 mg.

7. The composition of claim 2, wherein said guanosine analog antiviral agent is chosen from the group consisting of acycloguanosine, valacyclovir, penciclovir, famciclovir, and ganciclovir.

8. The composition of claim 2, wherein said guanosine analog antiviral agent is present in the amount of 50 mg to 400 mg.

9. The composition of claim 2, wherein said Vitamin D is chosen from the group consisting of Vitamin D2 and Vitamin D3.

10. The composition of claim 2, wherein said Vitamin D is present in the amount of 50 I.U. to 1000 I.U.

11. The composition of claim 2, wherein said composition is in a single oral dosage form chosen from the group consisting of a pill, capsule, and tablet.

12. The composition of claim 1, wherein said composition is further defined as 100 mg minocycline, 20 mg atorvastatin, 200 mg acycloguanosine, and 400 IU Vitamin D3 in a single dosage form.

13. A method of treating chronic inflammation, including the steps of:
administering a synergistically effective amount of a composition including an antibiotic, a lipophilic potentiating agent, a guanosine analog antiviral agent, and Vitamin D to an individual suffering from chronic inflammation; and
treating chronic inflammation.

14. The method of claim 13, wherein said administering step is further defined as administering the composition once every 12 hours in a single oral dosage form.

15. The method of claim 13, wherein said administering step is further defined as administering the composition for at least 45 days.

16. The method of claim 13, wherein the composition is further defined as a tetracycline antibiotic, a lipophilic statin, a guanosine analog antiviral agent, and Vitamin D in synergistically effective amounts.

17. The method of claim 16, wherein said tetracycline antibiotic is chosen from the group consisting of minocycline, tetracycline, chlortetracycline, oxytetracycline, demeclocycline, methacycline, tigecycline, and doxycycline.

18. The method of claim 16, wherein said tetracycline antibiotic is present in the amount of 25 mg to 500 mg.

19. The method of claim 16, wherein said lipophilic statin is chosen from the group consisting of atorvastatin, lovastatin, simvastatin, cerivastatin, fluvastatin, and mevastatin.

20. The method of claim 16, wherein said lipophilic statin is present in the amount of 5 mg to 40 mg.

21. The method of claim 16, wherein said guanosine analog antiviral agent is chosen from the group consisting of acycloguanosine, valacyclovir, penciclovir, famciclovir, and ganciclovir.

22. The method of claim 16, wherein said guanosine analog antiviral agent is present in the amount of 50 mg to 400 mg.

23. The method of claim 16, wherein said Vitamin D is chosen from the group consisting of Vitamin D2 and Vitamin D3.

24. The method of claim 16, wherein said Vitamin D is present in the amount of 50 I.U. to 1000 I.U.

25. The method of claim 16, wherein the composition is further defined as 100 mg minocycline, 20 mg atorvastatin, 200 mg acycloguanosine, and 400 IU Vitamin D3 in a single dosage form.

26. The method of claim 13, wherein said treating step includes at least one steps chosen from the group consisting of softening accumulations of synovial pannus, reducing amounts of synovial pannus, increasing nutrient supply to cartilage, restoring articulation and functional recovery of limbs affected by the chronic inflammation, reducing and/or eliminating pain, reducing and/or eliminating stiffness in joints, reducing and/or eliminating swelling, absorbing rheumatoid nodules, and combinations thereof.

27. The method of claim 13, wherein said treating step is further defined as eliminating and eradicating capsids immortalized by inflammatory processes and inducing a regenerative process of injured areas.

28. The method of claim 13, wherein said treating step further includes the step of reducing the individual's Expanded Disability Status Scale (EDSS) value.

29. The method of claim 13, wherein the chronic inflammation is due to rheumatoid arthritis.

30. The method of claim 13, further including the steps of removing residues at the intracellular level of mononucleosis and related strains, restoring the viscosity of synovial fluid and reducing thickened synovial membrane, thereby reducing joint inflammation, eliminating latent viral potential by eradicating viral caspids, and fixing calcium in the bones instead of being absorbed in the synovial pannus.

31. A method of reducing and/or eliminating symptoms of chronic inflammation, including the steps of:
administering a synergistically effective amount of a composition including an antibiotic, a lipophilic potentiating agent, a guanosine analog antiviral agent, and Vitamin D to an individual suffering from chronic inflammation; and
reducing and/or eliminating the individual's symptoms of chronic inflammation.

32. The method of claim 31, wherein the composition is further defined as tetracycline antibiotic, a lipophilic statin, a guanosine analog antiviral agent, and Vitamin D.

33. The method of claim 32, wherein the composition is further defined as 100 mg minocycline, 20 mg atorvastatin, 200 mg acycloguanosine, and 400 IU Vitamin D3 in a single dosage form.

34. The method of claim 31, wherein the symptoms reduced and/or eliminated are chosen from the group consisting of stiffness in joints, pain, swelling, and combinations thereof.

35. The method of claim 31, wherein the chronic inflammation is due to rheumatoid arthritis.

36. A method of recovering mobility of an individual suffering from chronic inflammation, including the steps of:
administering a synergistically effective amount of a composition including an antibiotic, a lipophilic potentiating agent, a guanosine analog antiviral agent, and Vitamin D to the individual; and
recovering mobility.

37. The method of claim 36, wherein the composition is further defined as tetracycline antibiotic, a lipophilic statin, a guanosine analog antiviral agent, and Vitamin D.

38. The method of claim 37, wherein the composition is further defined as 100 mg minocycline, 20 mg atorvastatin, 200 mg acycloguanosine, and 400 IU Vitamin D3 in a single dosage form.

39. The method of claim 34, wherein said recovering step further includes a step chosen from the group consisting of recovering the ability to move joints, recovering motor control, reducing and/or eliminating stiffness in the joints, reducing and/or eliminating pain, reducing and/or eliminating swelling, and combinations thereof.

40. The method of claim 36, wherein the chronic inflammation is due to rheumatoid arthritis.
